(19) United States
(12) Patent Application Publication
(10) Pub. No.: US 2024/0008833 A1
(43) Pub. Date: Jan. 11, 2024

(54) METHOD AND SYSTEM FOR EXECUTING AN IMAGE-BASED TASK

(71) Applicant: Siemens Healthcare GmbH, Erlangen (DE)

(72) Inventors: Elisabeth Preuhs, Erlangen (DE); Sai Gokul Hariharan, Forchheim (DE); Markus Kowarschik, Nürnberg (DE)

(21) Appl. No.: 18/218,187

(22) Filed: Jul. 5, 2023

(30) Foreign Application Priority Data
Jul. 5, 2022 (DE) 10 2022 206 846.7

(51) Int. Cl. A61B 6/00; G06T 7/10; G06T 7/20; G06T 7/00; G16H 30/40; G16H 40/63

(52) U.S. Cl. CPC A61B 6/545; G06T 7/10; G06T 7/20; G06T 7/0012; G16H 30/40; G16H 40/63; G06T 2207/10116; G06T 2207/10088; G06T 2207/10132; G06T 2207/30168

(57) ABSTRACT

To improve execution of image-based tasks, such as obtaining an x-ray image with a desired contrast while using a minimum x-ray dosage, a method in which one or more acquisition parameters of an image acquisition apparatus are automatically set as a function of the task is provided. An image of a biological tissue is acquired using the image acquisition apparatus for which one or more acquisition parameters have been automatically set. The task is executed based on the image of the biological tissue.

This application claims the benefit of German Patent Application No. DE 10 2022 206 846.7, filed on Jul. 5, 2022, which is hereby incorporated by reference in its entirety.

### BACKGROUND

The present embodiments relate to a method for executing an image-based task. The present embodiments further relate to a corresponding system for executing such an image-based task. The present embodiments also relate to a computer program and an electronically readable data medium.

Image management (e.g., using x-ray images) is an integral part of the decision process in active medical interventions, particularly in highly complex or time-critical cases. Following the acquisition and real-time processing of the image data, the visualization of the data allows the physician to assess the current status or progress of the intervention. During such procedures, the physician is usually situated at the side of the patient table in order to interact with the devices involved in the procedure. For example, the physician interacts with the imaging system or the medical devices or instruments that are used during the treatment. In this way, the physician may adapt the parameters of the image recording and the position of a specific device in order to achieve the best possible outcome for the patient.

A core component of all these methods is the navigation of the various devices to the positions at which treatment is required. The treatment depends on the anatomical region of interest (e.g., brain, liver, heart) and the underlying clinical condition of the patient. Many devices exist for different applications and treatments of various anatomical regions. Examples include guidewires, guide catheters, stents, and/or balloon catheters, aspiration catheters, coil and stent retrievers, etc.

In order to protect the physician from radiation and allow reliable and precise navigation of the device, such interventions may be assisted by active vascular robotics (e.g., the Corindus system). Here, the physician controls the devices from outside the operating theater with the aid of a joystick console. This type of intervention makes it possible to automate parts of the device movement and provide further assistance to the physician. One example is the automated guidance of a device from a starting position to a target position (e.g., a lesion that is to be treated). In this case, the available live images are to be processed by devices in order to detect and track the device positions so that the next movement/robot instruction may be derived therefrom.

As soon as the devices are tracked and moved automatically, it is no longer necessary to adapt the image quality so that the physician may analyze the current live images in order to decide the next movement step of the device. The x-ray dosage may therefore be reduced. The reduction in the x-ray dosage may decrease the harmful effects of the radiation exposure on the patient. However, the image quality is affected and possibly decreased thereby. Even with a reduced dosage, the image quality may nonetheless be adequate for the subsequent automatic processing.

The reduction in the dosage is usually controlled manually by the operator by setting various image recording parameters. The image quality resulting from these settings may not be optimal for automatic image processing tasks (e.g., device detection and tracking). Further, a constant dosage is usually set for the whole sequence.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, execution of image-controlled tasks is further optimized.

According to the present embodiments, provision is therefore made for a method for executing an image-based task. An image-based task consists in performing one or more work steps based on an image. The task or the work steps to be executed are therefore performed as a function of the image. The image-based task may be, for example, a therapeutic task, a data processing task, a robotic task, or similar.

In an act of the method, one or more acquisition parameters of an image acquisition apparatus are automatically set as a function of the task. An image acquisition apparatus such as, for example, an x-ray device, an MRT device, a sonography device, or similar is therefore provided. This image acquisition apparatus allows an image to be acquired as a function of a setting parameter or acquisition parameter (or a plurality thereof). In the present case, this acquisition parameter is set automatically. For this purpose, use is made of, for example, a predetermined algorithm. The automatic setting is effected as a function of the aforementioned task. For example, the predetermined algorithm therefore uses the defined task as an input parameter.

In a next act of the method, an image of a biological tissue is acquired using the image acquisition apparatus having one or more acquisition parameters that have been automatically set (if reference is made to an individual acquisition parameter in the following, a plurality of acquisition parameters may also be meant). The biological tissue is human, animal, or plant tissue, for example. If applicable, an instrument for therapeutic treatment is located in the tissue or in the tissue section that is to be acquired. Such instruments (e.g., catheters or similar) are mentioned in the introduction. Accordingly, the image is an x-ray image, an MRT recording, or similar. Since the image acquisition apparatus is set automatically as a function of the task, the acquisition of the image is effected as a function of the task accordingly.

Following thereupon, the task is executed based on the image of the biological tissue. If the task consists in guiding a catheter, for example, the catheter is guided in the biological tissue as a function of the image that has been acquired. Alternatively, if the task consists in effecting a segmentation in an image, the corresponding image processing for the segmentation is performed based on the image that was acquired using the automatic setting. It is thus possible to effect the widest variety of tasks with reference to an image, the acquisition of which was set automatically in a task-specific manner. It is thereby advantageously possible to optimize the execution of an image-based task with respect to many different considerations.

According to an embodiment variant, provision is made for the image acquisition apparatus to include an x-ray arrangement, for the acquisition parameter to be an x-ray dosage of the x-ray arrangement, and for the automatic setting to include setting the x-ray dosage. As mentioned in the introduction, the x-ray dosage is a critical parameter in the case of tissue examinations. The x-ray dosage should be kept to a minimum. It is therefore important to adapt the x-ray dosage (e.g., to the task that is subsequently to be executed). For example, a higher x-ray dosage is to be provided for a high image resolution than is required for a lower resolution. Likewise, it is possible to work with a lower x-ray dosage when tracking slow movements of instruments than in the case of faster movements. Thus, the x-ray dosage may be adapted automatically to the task that is to be executed at the time.

Similarly, provision may be made for the image acquisition apparatus to include a magnetic resonance arrangement, for the acquisition parameter to be a magnetic field parameter of the magnetic resonance arrangement, and for the automatic setting to include setting the magnetic field parameter. For example, the sampling rate may be decreased when a required image resolution is lower in comparison with high-resolution images. As a result of reducing the sampling rate, it is possible to save time in some circumstances. It is therefore advantageous to automatically adapt the respective magnetic field parameter to the task that is to be performed.

In a further alternative embodiment variant, provision may be made for the image acquisition apparatus to include a sonography arrangement, for the acquisition parameter to be an ultrasound parameter of the sonography arrangement, and for the automatic setting to include setting the ultrasound parameter. Here, likewise, it may be advantageous to automatically adapt a respective ultrasound parameter to the desired task. For example, it is likewise possible here to derive time advantages if the sampling is varied according to the task.

According to a further embodiment variant of the method, the task involves image processing. In the context of image processing, parameters such as the signal-to-noise ratio, the contrast, or similar may be of importance. Depending on these parameters, it may be desirable to set the acquisition parameter automatically. For example, a low x-ray dosage is set if only a low signal-to-noise ratio is required. Conversely, if applicable, a higher x-ray dosage is automatically set if this is required for the signal-to-noise ratio. The same applies, for example, to the magnetic field parameter or the ultrasound parameter in order to obtain corresponding time advantages, for example.

In a development of the method, the image processing may include a segmentation of image objects and/or a movement prediction. If the task consists, for example, in segmenting image objects very accurately, a high x-ray dosage or a high sampling rate may be required accordingly. The same applies to a movement prediction. If the image processing includes a movement prediction, it may be necessary to set (e.g., increase) the x-ray dosage, the sampling rate, or a correspondingly different parameter in a suitable manner in order to increase the accuracy. If the prediction of the movement of, for example, a catheter in the tissue is less critical, the x-ray dosage may possibly be reduced.

In a further embodiment, provision may be made for the method to be performed multiple times. A first processing value, relating to a quality or quantity of the task or part of the task, is generated during a first performance, and the first processing value is used for a second performance of the method. The use of the processing value from the first performance may provide that the processing value is compared with a threshold value, and the result of the comparison is used for the second performance (e.g., for the automatic setting). The processing value may, however, also be used in an optimization algorithm that acts on the second performance (and possibly further performances). In this way, the quality or quantity of the execution of the task may be retained or optimized for future executions of the task. The processing value in relation to the quality may be an approval value or an uncertainty value. A high quality may therefore be achieved with a high approval value or a low uncertainty value. The processing value in relation to the quantity may refer, for example, to a movement prediction. If a high movement value is predicted, for example, in the first performance of the method, this prediction may be used, for example, in the second performance of the method in order, for example, to increase the x-ray dosage or the sampling value accordingly. It is therefore already possible in a second repetition of the method to achieve an improved image quality or movement resolution.

According to a specific development of the method, the acquisition parameter may be set such that a respective processing value referring to the quality or quantity of the task or part of the task lies above or below a predetermined threshold value. For example, the acquisition parameter may be automatically set by closed-loop or open-loop control such that the respective processing value lies in a predetermined value range. The acquisition parameter therefore lies above and/or below respective threshold values. For example, the acquisition parameter may be set such that the uncertainty for a segmentation lies below a predetermined threshold value. The acquisition parameter may therefore be set automatically such that the task to be performed achieves the desired quality or quantity.

In an embodiment of the method, provision is made for the x-ray dosage as an acquisition parameter to be minimized to the extent that the respective processing value lies above or below the predetermined threshold value. The primary objective is, for example, therefore, to minimize the x-ray dosage. The x-ray power or the pulse width of an x-ray device may be considered as specific acquisition parameters. For example, the x-ray power may be reduced to such an extent that the processing value (e.g., quality) lies only just above a predetermined value. It is thereby possible to effect an optimization of the acquisition parameter. The optimization is tailored to the respective task.

In an alternative specific embodiment, for example, relating to the acquisition of an image by an x-ray arrangement, an MRT arrangement, or a sonography arrangement, provision may be made for the acquisition time, as the acquisition parameter or as one acquisition parameter of the plurality of acquisition parameters, to be minimized to the extent that the respective processing value lies above or below the predetermined threshold value. Specifically, the sampling rate may be reduced to the extent that the quality of an image lies only just above a specified threshold value. The acquisition time may thus be optimized in a task-specific manner.

In a further embodiment of the method, an image of the tissue may be obtained at regular time intervals using a predetermined setting of the acquisition parameter in each case, while for one or more images that are acquired between the intervals, the respective acquisition parameter is automatically set as a function of the respective task. For example, a high-resolution image may be obtained at fixed intervals (e.g., time intervals), while the images between these are obtained with a lower resolution if so allowed by the task. This provides that a high-resolution image may be obtained using a high x-ray dosage at fixed time intervals, but the images are acquired using a lower x-ray dosage in the meanwhile. It is thereby possible to reduce the overall x-ray dosage. The same also applies to other acquisition parameters and other acquisition apparatuses.

Also provided according to the present embodiments is a system for executing an image-based task, including an image acquisition apparatus that is configured to automatically set an acquisition parameter as a function of the task and to acquire an image of a biological tissue using the automatically set acquisition parameters(s), and a processing apparatus that is configured to execute the task based on the image of the biological tissue.

The advantages and possible variations described above in connection with the method of the present embodiments apply analogously to the system of the present embodiments. The individual method features may be considered as functional features in each case.

According to a further aspect of the present disclosure, provision is made for a computer program that may be loaded directly into a memory of a control arrangement of a system as described above, including program means for executing the acts of the method cited above when the program is executed in the control arrangement of the system. Provision is likewise made for an electronically readable data medium on which is stored electronically readable control information that includes at least one computer program of the type cited above, and is configured to perform a method of the aforementioned type when the data medium is used in a control arrangement of the above cited system. For example, the system may include a computer, a microcontroller, or an integrated circuit. Alternatively, the system may include a real or virtual combination (e.g., cluster) of computers. In this embodiment, the system includes a processor or a computing unit. The computing unit may include hardware elements and/or software elements (e.g., a microprocessor or a field programmable gate array (FPGA). A memory unit may take the form of non-permanent working memory (e.g., RAM) or permanent mass storage (e.g., hard disk, USB stick, SD card (SSD)).

### BRIEF DESCRIPTION OF THE DRAWINGS

FIG. 1 shows a schematic flow diagram of an embodiment of a method; and

FIG. 2 shows a schematic depiction of an embodiment of a system.

### DETAILED DESCRIPTION

The embodiments described in greater detail below represent embodiment variants of the present invention.

Purely by way of example, described below in greater detail is an automated system that may control the dosage setting as a function of the requirements of the subsequent image processing application, in order to achieve an image quality that is adequate for this application while using the smallest possible x-ray dosage. This example may be transferred analogously to other systems such as for example an MRT system, a sonography system, etc. For example, a magnetic field frequency or a sampling rate is then set as an acquisition parameter instead of the x-ray dosage. Likewise, a different image-based task than the image processing application may be selected.

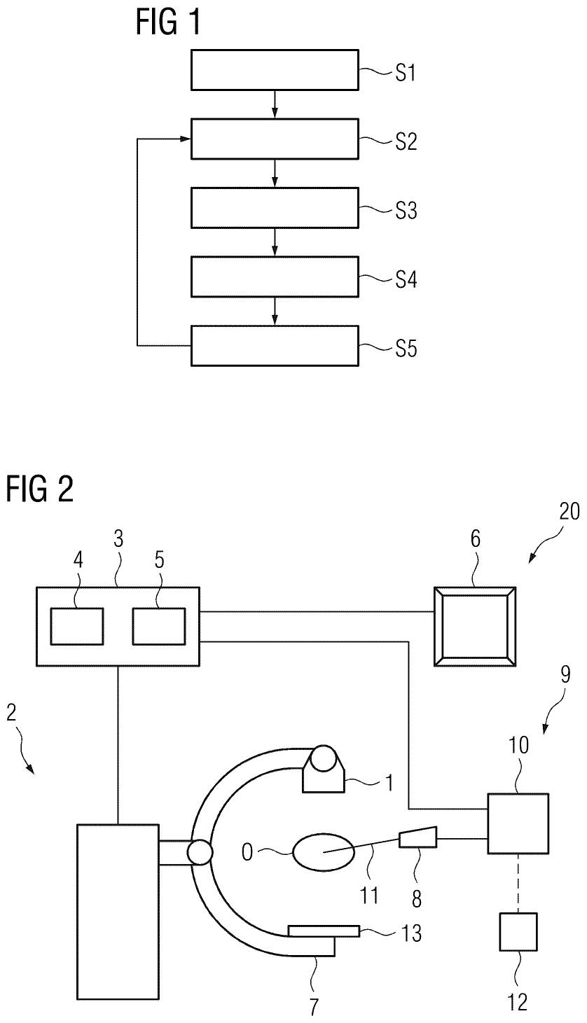

In a schematic block diagram, FIG. 1 shows individual method acts during the execution of a method according to an embodiment. A task is first predetermined in act S1. This task may include segmenting objects in an image. For example, it may be necessary to detect a catheter tip for the purpose of tracking. Any other chosen tasks (e.g., an image display with minimum signal-to-noise ratio or similar) may be provided as tasks.

Depending on this task that is predetermined or defined in act S1, an automatic setting of one or more acquisition parameters of an image acquisition apparatus takes place in act S2 as a function of the task. For example, an x-ray dosage of an x-ray apparatus is set as an acquisition parameter.

Using the setting that has been made, an image of a biological tissue is acquired according to act S3. This image may also show an instrument that has been inserted into the tissue, for example.

In a next act S4, the task is executed based on the image of the biological tissue that was acquired according to act S3. For example, optimizing a contrast, optimizing the signal-to-noise ratio, obtaining the position of a catheter head, or similar, is performed as a task.

In act S5, it is optionally possible to provide a processing value that was obtained when executing the task in act S4. For example, a quality value or a quantity value may be provided as a processing value. This processing value may then be used for act S2 in order to automatically set the acquisition arrangement. For example, optimization of a quality parameter may be effected in this way. For example, the adjustment of the setting parameter may be effected such that the quality value or quantity value lies above or below a threshold value or is situated in a value range.

In a specific exemplary embodiment, provision may be made for an image processing pipeline that automatically adapts the x-ray dosage settings such that the image quality for the subsequent automated tasks (e.g., device detection in order to decide the next device maneuver) remains sufficient but requires the lowest dosage possible.

A possible implementation may the following four acts: 1) The first image (e.g., series) is recorded using an initial dosage. 2) The automated image processing task (e.g., device detection) is performed for the first image (e.g., series), this containing the results (e.g., device segmentations of the current image, allowing for the segmentations of the preceding images) and an uncertainty evaluation (e.g., processing value) that describes how accurate the results of the algorithms are. An analysis of the results (e.g., segmentations) of the current image and of the preceding images is performed in order to identify any regions with movement. The movement is compensated, and the image sequences are then processed together in order to improve the signal-to-noise ratio in the x-ray images. 3) The recording of further image data takes place with a reduction (e.g., further reduction) of the x-ray dosage (e.g., by decreasing the x-ray power and/or the pulse width) for as long as the uncertainty value lies below a specified threshold value (e.g., the automatic image processing may be performed with sufficient accuracy). 4) When the uncertainty value is higher than a threshold value, the x-ray dosage is not reduced further (e.g., the minimum dosage for this task has been found). Further data acquisitions are then performed using this dosage value.

In a particular embodiment variant, some of the images (e.g., every n-th image) may be a) recorded using the preferred image quality settings of an operator (e.g., physician), and/or b) processed using the preferred image processing settings of the operator, such that the operator may, for example, check whether the automated intervention is being executed correctly, or may check the current status of the method manually using their normal settings.

In another embodiment variant, the x-ray dosage (e.g., x-ray power and/or pulse width) is reduced image by image if the segmentations are sufficiently accurate and if less movement is predicted based on the analysis of the preceding images and/or in consideration of the method that is being performed. As soon as a significant movement is predicted, for example, the dosage is gradually increased, together with the image rate, until the uncertainty value reaches the required threshold value. This provides that the dosage may be modulated during the overall sequence. The required image quality is maintained, and the radiation exposure is reduced for the patient.

FIG. 2 shows a complete system 20 for medical use, having, for example, an x-ray device 2 with a C-arm 7, and a robot system 9. Arranged on the C-arm 7 of the x-ray device 2 are an image sensor in the form of an x-ray detector 13 and an x-ray source 1. The C-arm 7 may be positioned in a plurality of directions, for example. A control unit 3 is provided for the purpose of controlling the x-ray device 2. The robot system 9 has a robot control unit 10 that controls a drive system 8 for navigating an instrument (e.g., a catheter 11) in an organ O (e.g., a hollow organ) of a patient. The robot control unit 10 is capable of receiving input instructions from an input unit 12. The robot system 9 is configured for the navigation of an instrument in a hollow space of a patient, for example, where the navigation may be performed robotically or with robotic assistance and is image-monitored using the x-ray device 2.

The method of the present embodiments may be performed by the complete system 20. The x-ray detector 13 is configured to record image sensor raw data. The complete system may also have a processing unit 4 for processing raw data from the image sensor in order to provide a first output image dataset that is configured for human use, and/or a second output image dataset that is configured for further processing by automated means. The first output image dataset may be displayed to a user on an optional display unit 6 (e.g., monitor). A unit 5 for automated further processing of the second output image dataset (e.g., using a machine-learning algorithm) may also be provided. The data that has undergone further processing may then be used to control the navigation.

In addition to the x-ray system (e.g., 2D x-ray system, 3D x-ray system, CT), the method may also be used for other imaging systems (e.g., MRT, ultrasound device, or SPECT).

The method or system of the present embodiments may advantageously set the x-ray dosage or other acquisition parameter automatically (e.g., such that it is optimal or, if applicable, as low as possible) during live operation for the next task (e.g., image processing task). The automated tasks (e.g., for image processing and/or device manipulation) therefore define the acquisition parameters (e.g., optimal image recording parameters and image processing software parameters). This provides that the x-ray dosage or the acquisition parameter(s) do not have to be set manually.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for executing an image-based task, the method comprising:
automatically setting one or more acquisition parameters of an image acquisition apparatus as a function of the image-based task;
acquiring, by the image acquisition apparatus with the automatically set one or more acquisition parameters, an image of a biological tissue; and
executing the image-based task based on the acquired image of the biological tissue.

2. The method of claim 1, wherein the image acquisition apparatus comprises an x-ray arrangement,
wherein the one or more acquisition parameters include an x-ray dosage of the x-ray arrangement, and
wherein the automatic setting of the one or more acquisition parameters comprises setting the x-ray dosage.

3. The method of claim 1, wherein the image acquisition apparatus comprises a magnetic resonance arrangement,
wherein the one or more acquisition parameters include a magnetic field parameter of the magnetic resonance arrangement, and
wherein the automatic setting of the one or more acquisition parameters comprises setting the magnetic field parameter.

4. The method of claim 1, wherein the image acquisition apparatus comprises a sonography arrangement,
wherein the one or more acquisition parameters include an ultrasound parameter of the sonography arrangement, and
wherein the automatic setting of the one or more acquisition parameters comprises setting the ultrasound parameter.

5. The method of claim 1, wherein the image-based task comprises image processing.

6. The method of claim 5, wherein the image processing comprises a segmentation of image objects, a movement prediction, or the segmentation of the image objects and the movement prediction.

7. The method of claim 1, wherein the method is performed multiple times,
wherein a first processing value relating to a quality or quantity of the image-based task or part of the image-based task is generated during a first performance of the method, and
wherein the first processing value is used for a second performance of the method.

8. The method of claim 7, wherein the one or more acquisition parameters are set, such that a respective processing value relating to the quality or quantity of the image-based task or the part of the image-based task lies above or below a predetermined threshold value.

9. The method of claim 8, wherein the image acquisition apparatus comprises an x-ray arrangement,
wherein the one or more acquisition parameters include an x-ray dosage of the x-ray arrangement,
wherein the automatic setting of the one or more acquisition parameters comprises setting the x-ray dosage, and
wherein the x-ray dosage as an acquisition parameter of the one or more acquisition parameters is minimized such that the respective processing value lies above or below the predetermined threshold value.

10. The method of claim 8, wherein the image acquisition apparatus comprises an x-ray arrangement,
wherein the one or more acquisition parameters include an x-ray dosage of the x-ray arrangement,
wherein the automatic setting of the one or more acquisition parameters comprises setting the x-ray dosage, and
wherein the one or more acquisition parameters include acquisition time, the acquisition time being minimized to the extent that the respective processing value lies above or below the predetermined threshold value.

11. The method of claim 8, wherein the image acquisition apparatus comprises a magnetic resonance arrangement,
wherein the one or more acquisition parameters include a magnetic field parameter of the magnetic resonance arrangement,
wherein the automatic setting of the one or more acquisition parameters comprises setting the magnetic field parameter, and
wherein the one or more acquisition parameters include acquisition time, the acquisition time being minimized to the extent that the respective processing value lies above or below the predetermined threshold value.

12. The method of claim 8, wherein the image acquisition apparatus comprises a sonography arrangement,
wherein the one or more acquisition parameters include an ultrasound parameter of the sonography arrangement,
wherein the automatic setting of the one or more acquisition parameters comprises setting the ultrasound parameter, and
wherein the one or more acquisition parameters include acquisition time, the acquisition time being minimized to the extent that the respective processing value lies above or below the predetermined threshold value.

13. The method of claim 7, wherein an image of the tissue is obtainable in each case at regular time intervals using a predetermined setting of the one or more acquisition parameters, while for one or more images which are acquired between said intervals, the respective acquisition parameter is automatically set as a function of the respective task (S2).

14. A system for executing an image-based task, the system comprising:
an image acquisition apparatus (2) which is designed to automatically set (S2) one or more acquisition parameters as a function of the task and to acquire an image (S3) of a biological tissue using the automatically set acquisition parameter(s), and
a processing apparatus which is designed to execute the task (S4) on the basis of the image of the biological tissue.

15. An electronically readable data medium on which is stored electronically readable control information that comprises at least a computer program as claimed in claim 13 and is configured to perform a method as claimed in one of claims 1 to 11 when the data medium is used in a control arrangement of a system as claimed in claim 12.
